(19) United States
(12) Patent Application Publication
(10) Pub. No.: US 2015/0320783 A1
(43) Pub. Date: Nov. 12, 2015

(54) N-METHYLSEROTONIN AND RELATED SUBSTANCES FOR USE IN TREATING / LESSENING THE OCCURRENCE OF HOT FLASHES RELATED TO MENOPAUSE

(71) Applicant: DSM IP ASSETS B.V., Heerlen (NL)

(72) Inventors: Christopher Michael BUTT, Kaiseraugst (CH); Michael WEISER, Kaiseraugst (CH)

(21) Appl. No.: 14/761,834

(22) PCT Filed: Jan. 24, 2014
(86) PCT No.: PCT/IB2014/058524

§ 371 Date: Jul. 17, 2015

Related U.S. Application Data
(60) Provisional application No. 61/756,529, filed on Jan. 25, 2013.

(51) Int. Cl. A61K 31/7056; A23L 1/30; A61K 36/758

(52) U.S. Cl. CPC A61K 31/7056; A61K 36/758; A23L 1/3002; A23V 2002/00

(57) ABSTRACT

Zanthoxylum piperitum (commonly known as Japanese pepper) seeds, including its mature seeds, contain the NMS precursor/V-methylserotonin 5-O-B-glucoside (“NMS-glucoside”) present in sufficient concentration and are metabolized with sufficient efficiency so as to provide an effective amount of NMS for use in amelioration of menopausal symptoms, including hot flashes/flushes. The Japanese pepper seeds or Japanese pepper seed extracts can also be used to enrich other sources of NMS or NMS precursors in order to provide a nutraceutical or food supplement for controlling hot flashes.

### BRIEF DESCRIPTION OF THE INVENTION

This invention relates to the use N-methylserotonin glucoside (“NMS-glucoside”) and/or N-methylserotonin (NMS), or other NMS precursors in the treatment, prevention, or amelioration of hot flashes (also called hot flushes) related to menopause. The NMS-glucoside may be obtained from a plant source, such as Zanthoxylum piperitum seeds or seed extracts. It also relates to the use of various plant extracts, including black cohosh, which have been fortified with at least one of the following: N-methylserotonin (“NMS”), NMS-glucoside, Zanthoxylum piperitum seeds, Zanthoxylum piperitum seed extract containing, NMS-glucoside, other NMS precursors, or mixtures thereof.

### BACKGROUND OF THE INVENTION

Black cohosh (known as both Actaea racemosa and Cimicifuga ramcemosa) extracts contain many nutrients that are not understood completely. Nonetheless, humans have used black cohosh for centuries based upon anecdotal and limited clinical evidence that it alleviates menopause-related hot flashes and improves mood. One of the nutrients in black cohosh, N-methylserotonin (NMS), is interesting because existing data for this ingredient lend some support to claims that certain Black cohosh extracts may be useful for ameliorating hot flashes. See, e.g. Powell et al 2008 J. Agric. Food Chem. 56:11718.

Serotonin (5-HT) receptors have been implicated in thermoregulation (specifically the 5-HT₇ isoform; FIG. 1A), and clinical trials in humans indicate that selective serotonin reuptake inhibitors (SSRIs) are effective in treating the symptoms of hot flashes). These findings demonstrate that serotonin signaling has some importance in thermoregulation. Interestingly, NMS binds preferentially to 5-HT₇ receptors over other 5-HT receptors, and it activates 5-HT₇ receptors with an affinity (EC₅₀=22 nM; FIG. 1B;) that is equivalent to that of serotonin itself. However, the likely concentration of NMS in a single black cohosh dose (˜30 ppm of 80 mg black cohosh extract) can only deliver 2.4 μg of NMS. This dose translates roughly to a maximum concentration of 0.18 nM in a 60-kg human female. This NMS concentration is high enough for binding to 5-HT₇, but it may not be sufficient for stand-alone functional effects at either 5-HT₇ (FIG. 1B) or the 5-HT transporter (SERT; FIG. 1C). Since the efficiency of black cohosh extraction may vary between production lots, this dosage issue may provide some explanation for the mixed results observed with black cohosh in clinical studies. Furthermore, black cohosh extracts are standardized to actein content, not NMS.

The dose of NMS necessary for mitigating menopausal symptoms is likely higher than what can be provided by black cohosh, thus there is a need in the art for a natural source of NMS which can be used to ameliorate menopausal symptoms such as hot flashes.

### DETAILED DESCRIPTION OF THE INVENTION

It has been found, in accordance with this invention that Zanthoxylum piperitum (commonly known as Japanese pepper) seeds, including its mature seeds, contain the NMS precursor N-methylserotonin 5-O-β-glucoside (“NMS-glucoside”) present in sufficient concentration and are metabolized with sufficient efficiency so as to provide an effective amount of NMS for use in amelioration of menopausal symptoms, including hot flashes/flushes. Thus, one aspect of this invention is the use of Japanese pepper seeds or Japanese pepper seed extracts which contain NMS-glucoside in food supplements or nutraceuticals for the use of controlling menopausal symptoms.

Thus:
a) Japanese pepper seeds or extracts of Japanese pepper seeds that contain NMS or NMS-glucoside, or mixtures thereof; or
b) NMS-glucoside
c) Other plant sources containing NMS or NMS-glucoside or mixtures thereof
d) Other NMS precursors that can be metabolized to NMS, regardless of origin can be used alone or in mixtures in effective amounts to lessen menopausal symptoms including hot flashes, or can be used in combination with NMS so that the combination is an effective amount to ameliorate hot flashes.

The NMS which can be used in this invention may be part of a plant preparation, such as an extract. A potential source is black cohosh, but Japanese pepper is the preferred source due to known higher concentrations of NMS precursors. Alternatively, synthetic NMS, regardless of source may be used.

Thus this invention also provides for
a) any plant extract, regardless of species, which contains a minimum effective dose of NMS, a precursor of NMS; or mixtures thereof;
b) use of a composition comprising a minimum effective does of NMS or a precursor of NMS, regardless of whether the NMS is derived from a plant extract;
c) use of plant extracts which naturally contain NMS or a precursor of NMS, but at a less than effective dosage amount, to which additional NMS or a precursor has been added in order to bring the total amount of NMS or precursor to the effective amount;
d) combinations of the above;
e) use of any of a-d, above, in combination with other ingredients useful for control of menopausal, peri-menopausal, and/or post-menopausal symptoms.

This invention also provides a method of preventing, treating, ameliorating, and/or decreasing the severity or occurrence of menopausal, peri-menopausal, and/or post-menopausal symptoms comprising:
administering an effective amount of an ingredient selected from the group consisting of: NMS-glucoside, NMS, Zanthoxylum piperitum seeds, Zanthoxylum piperitum seed extract comprising NMS-glucoside, NMS oxalate, NMS hydrochloride, NMS dihydrochloride; and mixtures thereof
to a woman experiencing said symptoms or to a woman who is menopausal, peri-menopausal, or post-menopausal.

This invention also relates to a mixture of plant extracts, preferably black cohosh and Japanese pepper, which together contain an effective amount of at least one active ingredient selected from the group consisting of NMS, an NMS precursor, or mixtures thereof.

Another aspect of this invention is an food supplement or nutraceutical wherein the only active ingredient effective against menopausal symptoms is NMS, a NMS precursor, or mixtures thereof.

The Japanese pepper, Zanthoxylum piperitum, which contains 50-fold more NMS (in the form of its precursors, such as NMS-glucoside) than black cohosh may also be used as part of this invention. Thus, Zanthoxylum could serve as a viable alternative source of NMS.

Another aspect of this invention is the use of NMS, NMS-glucoside or other NMS precursor (either alone or in admixture) as the sole ingredient targeting hot flashes in a nutraceutical or pharmaceutical composition.

Use of the Zanthoxylum extracts in combination with kudzu and soy extracts, or genistein is also envisioned.

### DEFINITIONS

As used herein, the following definitions apply:

“NMS precursor” is a compound which, after ingestion, is transformed into NMS. Specific compounds which are included in this definition are: NMS oxalate, NMS hydrochloride, NMS dihydrochloride and NMS glucoside. “Other NMS-precursors” refers to NMS precursors except NMS-glucoside.

“Prevention” is not meant to ensure that all possible hot flashes never occur. Rather it is used to indicate that the risk of experiencing hot flashes is reduced, the incidence is reduced, the severity is lessened, and/or in general, the discomfort associated with them the ameliorated. It may also be used to indicate that the time interval between experiencing the hot flashed is increased.

“Perimenopausal” a time prior to menopause (cessation of menstrual periods for at least 12 months), characterized by one or more of: estrogen levels rising/falling unevenly, menstrual cycles without ovulation, experience of menopausal symptoms such as hot flashes, sleep problems, mood changes, bone loss, or vaginal dryness although menstrual cycles are still experienced. This generally occurs in women in their 40's.

“Menopause” is the point when a woman no longer has menstrual periods. The ovaries have stopped producing eggs and producing most of their estrogen. It is diagnosed when a woman has gone without a menstrual period for 12 consecutive months.

“Postmenopause”—The time after menopause. A woman is considered postmenopausal when she has not had a menstrual period for an entire year.

### BRIEF DESCRIPTION OF THE FIGURES

FIG. 1. N-methylserotonin (NMS) could mitigate hotflashes via 5-HT₇ receptors and could improve mood by inhibiting serotonin uptake. A) 5-HT₇ receptors decreased body temperature when activated in wildtype (WT) animals, but a 5-HT₇ agonist did not decrease body temperature in 5-HT₇ knockout (KO) animals (Hedlund et al., 2004). B) Maximal activation of the 5-HT₇ receptor by NMS occurred at 50-100 nM with an EC₅₀ of ˜22 nM. C) Maximal inhibition of the serotonin reuptake transporter by NMS occurred at 1-10 M with an IC₅₀ of ˜500 nM (100-fold lower affinity than Prozac; Powell et al., 2008).

FIG. 2. Study design for evaluating the effects of dietary NMS on hot flashes and mood-related behaviors in an animal model of menopause. The animals were subjected to surgically induced menopause (ovariectomy; OVX) and fed diets that contained different levels of NMS. The animals were then tested for locomotor activity in the open field, anxiety-like behaviors on the elevated plus maze, and depression-like behaviors with the swim test. Hot flashes were then induced with intravenous (i.v.) calcitonin gene related peptide (CGRP) and skin temperature was monitored. Tissues were collected two hours after CGRP challenge. Abbreviations: s.c., subcutaneous; st., straight body weight conversion; BSA, body surface area conversion; BC 540, black cohosh commercial dose; cAMP, cyclic adenosine monophosphate; Vₘₐₓ, maximum velocity of an enzymatic reaction; SERT, serotonin transporter; IC₅₀, half-maximal inhibitory concentration; Iₘₐₓ, maximal inhibitory concentration.

FIG. 3. Representative raw and baseline-corrected data for skin temperature during experimentally induced hot flashes. Animals receiving no dietary treatment (0 ug/kg/d NMS) had the highest basal temperatures and the highest CGRP response above their calculated baselines. Animals receiving 0.48 or 18.5 ug/kg/d NMS through their diet exhibited lower basal temperatures and lower CGRP responses than the untreated controls. Animals receiving subcutaneous estradiol and no dietary NMS (positive controls) had the lowest basal temperatures and the lowest responses to CGRP.

FIG. 4. Dietary NMS reduced baseline skin temperature and blunted the CGRP-induced hot flash response. The group receiving 18.5 ug/kg/d NMS had decreased basal skin temperature in comparison to untreated controls. Similar effects of NMS on the hot flash response were detected when the baseline corrected data were analyzed with repeated measures ANOVA and when the areas under the curves were analyzed with one-way ANOVA. Treatment groups that did not have within-day controls associated with them were removed from the analyses. Statistical parameters are in the graphs. Error bars represent the standard errors of the means (SEM). Treatments that do not share common letters were significantly different (p<0.05) from one another in Tukey's post hoc test.

FIG. 5. N-methylserotonin (NMS) increased circulating (plasma) estradiol levels in ovariectomized female rats, which suggests that NMS modulated non-ovarian synthesis and/or secretion of 17β-estradiol. A) Dietary supplementation of NMS increased plasma estradiol in a dose-dependent manner. Estradiol supplemented animals had the highest levels of plasma estradiol. B) Within-day controlled data. ANOVA parameters with (upper) and without (lower) the estradiol supplemented group are inset within each graph. Treatments that do not share common letters were significantly different (p<0.05) from one another in Tukey's post hoc test. Error bars represent the SEM

FIG. 6. Dietary NMS did not alter body weight and was not uterotrophic in ovariectomized female rats. A) Mean body weight was not different between NMS supplemented groups and the control diet; estradiol implanted animals had significantly lower body weight throughout the experiment compared to all other groups. B and C) Weight change as a percentage of original weight was not different between dietary treatments, with the exception of estradiol implanted animals, when examined throughout or at the end of the study. D) NMS did not alter uterine weight after 80 days of supplementation; estradiol implanted animals had significantly higher uterine weights compared to ovariectomized controls and all NMS supplemented groups. Significant differences (p<0.05) between groups were determined by repeated measures ANOVA or 1-way ANOVA, where appropriate, followed by Tukey's post hoc tests (***, p<0.001). ANOVA parameters are inset within each graph where a significant main effect was detected. Error bars represent the SEM.

FIG. 7. Dietary NMS did not alter behavioral measures of mood in ovariectomized female rats. A) Estradiol implanted animals spent less time immobile (antidepressant-like behavior) during the second session of the forced swim test (FST; total test time 600 s). B) There were no significant differences amongst groups in time spent climbing during the second session of the FST. C) In the open field (test for anxiety-like behaviors and locomotion), there were no differences across groups in total distance traveled in the arena during the 30 minute test. D) Overall exploratory behavior in the elevated plus maze (test for anxiety-like behaviors) was low in all groups and there were no differences amongst groups for time spent in the open arms (anxiolytic behavior). Significant differences (p<0.05) between groups were determined by 1-way ANOVA followed by Tukey's post hoc tests (*, p<0.05). ANOVA parameters are inset within each graph where a significant main effect was detected. Error bars represent the SEM.

FIG. 8. Follow-up study design for evaluating the effects of dietary NMS-glucoside from milled Zanthoxylum seed on hot flashes and mood-related behaviors in an animal model of menopause. As with the first study (FIG. 2), the animals were subjected to surgically induced menopause (ovariectomy; OVX). In this second study, some animals were fed diets that repeated two doses of NMS, while other animals were fed milled Zanthoxylum seed to supply equivalent doses of NMS-glucoside. The animals were then tested for locomotor activity in the open field, anxiety-like behaviors on the elevated plus maze, and depression-like behaviors with the swim test. Hot flashes were then induced with intravenous (i.v.) calcitonin gene related peptide (CGRP) and skin temperature was monitored. Tissues were collected two hours after CGRP challenge.

FIG. 9. Dietary NMS-glucoside from milled Zanthoxylum seed blunted the CGRP-induced hot flash response in a manner similar to estradiol and NMS. A) Repeated measures ANOVA detected that animals receiving Zanthoxylum seed had decreased hot flash responses that were significantly different from untreated controls but equivalent to those of animals given estradiol or equivalent doses of chemically synthesized NMS oxalate. B) Similar effects on the hot flash response were detected when the areas under the curves were analyzed with one-way ANOVA. Statistical parameters are in the graphs. Error bars represent the standard errors of the means (SEM). Treatments that do not share common letters were significantly different (p<0.05) from one another in Tukey's post hoc test.

FIG. 10. Similar to the first study, the follow-up study demonstrated that Zanthoxylum seed and NMS did not alter behavioral measures of mood. No significant effects of treatment were detected in A) the open field (test for anxiety-like behaviors and locomotion), B) the elevated plus maze (test for anxiety-like behaviors), or C) the forced swim test (for depressive-like behaviors). Error bars represent the SEM.

FIG. 11. Dietary NMS-glucoside from Zanthoxylum seed was not uterotrophic in ovariectomized female rats but may have altered body weight. A) Similar to the first study, Zanthoxylum seed and NMS did not alter uterine weight, but animals receiving estradiol had, as expected, significantly higher uterine weights compared to ovariectomized controls and all other groups. B) Over time, weight change as a percentage of original weight was different between controls and pepper supplemented or NMS supplemented groups. Estradiol implanted animals had significantly lower body weight throughout the experiment compared to all other groups. C) However, as with the first study, overall weight change as a percentage of original weight was not different between dietary treatments, with the exception of estradiol implanted animals, when examined at the end of the study. Significant differences (p<0.05) between groups were determined by repeated measures ANOVA or 1-way ANOVA, where appropriate. Treatments that do not share common letters were significantly different (p<0.05) from one another in Tukey's post hoc tests. ANOVA parameters were removed from (B) and (C) for the sake of simplicity. Error bars represent the SEM.

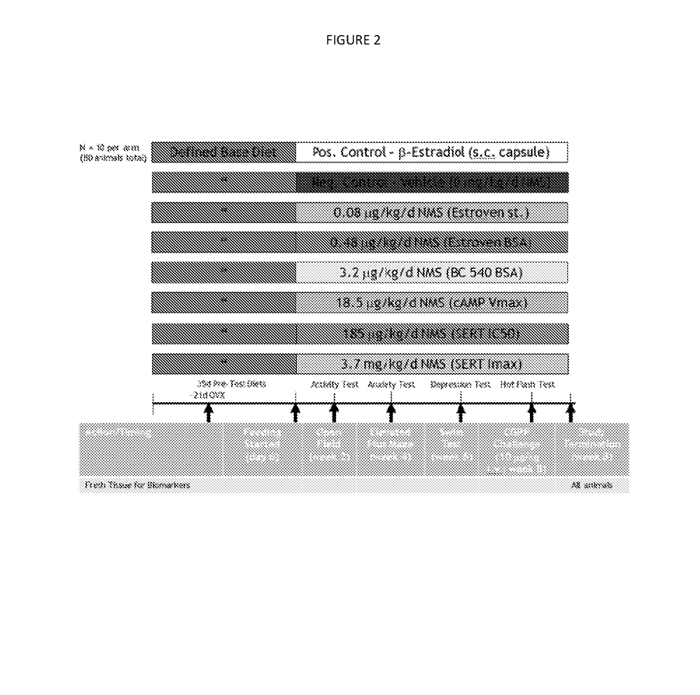

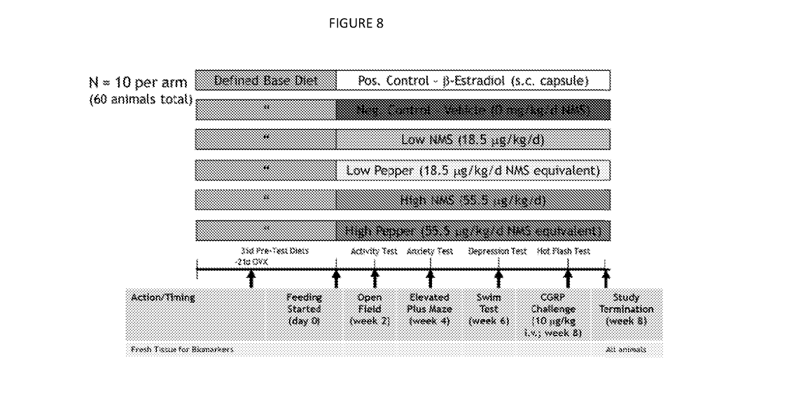

The studies reported here were focused on the effect of dietary NMS and NMS-glucoside on the hot flash response and on behaviors related to mood in an animal model of menopause. As the study designs in FIGS. 2 and 8 illustrate, the animals were subjected to surgically induced menopause and fed diets that contained different levels of NMS or NMS-glucoside. We tested the supplemented animals for locomotor activity in the open field, anxiety-like behaviors on the elevated plus maze, and depression-like behaviors with the swim test. We then experimentally induced a hot flash response and monitored changes in the animals' skin temperature. The overall objective of this work was to provide supporting data for NMS or NMS-glucoside as dietary supplements for use in menopausal women, or for women in the peri- or post-menopausal stages who are experiencing menopausal symptoms, or who are desirous of reducing the risk of experiencing such symptoms.

### Plant Sources of NMS, NMS-Glucoside or Other NMS Precursors

Plants which may be used as a sources of NMS, NMS-glucoside, and/or other NMS precursors include: Zanthoxylum species, such as Z. piperitum Z. simulans, Z. bungeanum, Z. schinifolium, Z. nitidum, Z. rhetsa, Z. alatum, Z. acanthopodium, and/or Z. americanum. The preferred source is Z. piperitum.

In addition, citrus plants are also known contain NMS precursors, so Citrus bergamia (bergamot), lemon, orange, mandarin orange, chinotto, citron may also be used. The compounds in citrus plants may be found in leaf, peel, endocarp and seeds.

Lichens of the genus Collema, including C. cristatum, C. callopismum, C. flaccidum, and C. fuscovirens may also be used.

Seeds may be prepared for use by simply crushing, milling or powdering. Extracts may be made in accordance with methods well-known in the art, e.g., by (an) extraction with solvents like methanol, ethanol, ethyl acetate, diethylether, n-hexane, methylene chloride, or with supercritical fluids like carbon dioxide (pure or in mixture with other solvents such as alcohols) or dinitrogen oxide, (b) hydrodistillation for obtaining essential oils or (c) extraction/distillation with hot gases like nitrogen.

Ingestion of the seeds according to this invention is to be distinguished from the mere ingestion of seeds as a spice or flavoring, such as is used in many Asian or Asian-inspired food dishes, as this invention contemplates use of the NMS-glucoside in the absence of accompanying foodstuffs. Thus, this invention specifically excludes the ingestion of NMS-glucoside or an NMS-glucoside precursor admixed with food, such as in a spicy meal or snack.

### Dosages

An “effective dose” of NMS which needs to be present in a daily dosage for an adult is at least 5 micrograms to 225 milligrams, preferably 50 micrograms to 100 milligrams; more preferably 100 micrograms to 75 milligrams. Alternatively, an effective dose is at least 5, 10, 25, 50, 100, 200, 250, 500, 750, 1000, 1500, 2000, 2500, 3000, 3500, 4000, or 4500 micrograms per day. Alternatively the amount is at least 500 micrograms per day, and preferably higher.

For Japanese Pepper seed, an “effective dose” is from 50-20000 mg seed per day; preferably from about 80 to 2500 mg seed per day or higher.

For NMS-glucoside, an “effective dose” which should be present in a daily dosage for an adult is at least 5 micrograms to 225 milligrams, preferably 50 micrograms to 100 milligrams; more preferably 100 micrograms to 75 milligrams. Alternatively, an effective dose is at least 5, 10, 25, 50, 100, 200, 250, 500, 750, 1000, 1500, 2000, 2500, 3000, 3500, 4000, 4500, 5000, or 5500 micrograms per day. Alternatively the amount is at least 600 micrograms per day, and preferably higher.

For other NMS-precursors, the effective amounts would be calculated as the amount of precursor which would be required to yield an effective amount of NMS (supra).

The NMS and NMS-glucoside content can be verified using liquid chromatography with mass spectrometric detection. Extraction methods can be performed similar to those of Yanase et al., 2010.

The daily doses of the active ingredients as set forth above may be taken as a single dose or may be taken in multiple smaller doses throughout the day, such as 2× per day, 3× per day or in another convenient dosage regime. Consumption on a regular basis is recommended, i.e. for at least a week, preferably for a least a month, and more preferably for a time period which can be characterized as “long term”, i.e. longer than one month's duration.

### Formulations

The dietary supplements and nutraceutical compositions according to the present invention may be in any galenic form that is suitable for administering to a human, especially in any form that is conventional for oral administration, e.g. in solid form, such as tablets, pills, granules, dragées, capsules, and effervescent formulations such as powders and tablets, or in liquid form such as solutions, emulsions or suspensions as e.g. beverages, pastes and oily suspensions. The pastes may be encapsulated in hard or soft shell capsules, whereby the capsules feature e.g. a matrix of (fish, swine, poultry, cow) gelatin, plant proteins or lignin sulfonate. Examples for other application forms are forms for transdermal, parenteral or injectable administration. The dietary supplements and nutraceutical compositions may be in the form of controlled (delayed) release formulations.

The dietary and nutraceutical compositions according to the present invention may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellyfying agents, gel forming agents, antioxidants and antimicrobials.

The following non-limiting Examples are presented to better illustrate the invention

### EXAMPLES

### Methods

The animals arrived at approximately 60 days of age, and were placed on specific diets throughout the study (to approximately 160 days of age). The diets were formulated and provided by Dyets Inc. (Bethlehem, Pa.) and are based on a modified AIN-93G diet. These diets were fed ad libitum. Two weeks after arrival, at approximately 80 days of age, the animals were ovariectomized, allowed to recover for three weeks, and then placed on defined diets. In the first study, these diets contained 0, 0.08, 0.48, 3.2, 18.5, 185, or 3700 μg/kg/day of NMS dihydrochloride (FIG. 2). In the second, follow-up study, the diets contained no NMS (control), NMS oxalate (18.5 or 55.5 μg/kg/d), or milled Zanthoxylum seed in quantities that supplied NMS-glucoside at equivalent doses (18.5 or 55.5 μg/kg/d; FIG. 8). The NMS and NMS-glucoside content was verified using liquid chromatography with mass spectrometric etection. Extraction methods were similar to those of Yanase et al., 2010.

All diets were fed for 2 weeks prior to the first behavioral assessment (open field). Positive control animals received a 5.0 mm subcutaneous silastic capsule implant packed with a crystalline 17β-estradiol and cholesterol mixture (25%, 0.058 inch inner diameter, 0.077 inch outer diameter; Dow Corning #508-006). Animals were then examined for locomotor activity (open field) and in two behavioral assays to assess mood (elevated plus maze and swim test) separated by two weeks in-between each assay. Two weeks after the final behavioral task (swim test), the animals were anesthetized and their physiological response to an experimentally-induced hot flash was tested. After the temperature monitoring was complete, tissue was harvested while the animals were still anesthetized.

Ovariectomy.

Two weeks after arrival, each animal was anesthetized via isoflurane inhalation and the dorsal mid-lumbar area was shaved and swabbed with surgical scrub, iodine and alcohol. The animal was then placed on a warm heat pad covered by a sterile cloth/pad in ventral recumbency with tail towards surgeon. A 2-3 cm dorsal midline skin incision was made halfway between the caudal edge of the ribcage and the base of the tail. A single transverse incision of 5.5-10 mm long was made into the muscle wall on both the right and left sides approximately ⅓ of the distance between the spinal cord and the ventral midline. The ovary and the oviduct were exteriorized through the muscle wall. A hemostat was clamped around the uterine vasculature between the oviduct and uterus and the uterine vasculature was ligated with dissolvable suture. Each ovary and part of the oviduct was removed with single cuts through the oviducts near the ovary. The hemostat was removed, and the remaining tissue replaced into the peritoneal cavity. The ovary on the other side was removed in a similar manner. The muscle incision is not sutured. Sterile wound clips were used to close the skin incision. Animals were monitored daily for signs of infection or morbidity.

Animal Handling.

Rats were handled each day for four days prior to behavioral analysis. This involved gently removing the animal from its cage and holding it in the experimenter's lap on a towel without restraint, allowing the animal to explore. This acclimatized the animal to handling by experimenters. All behavioral testing occurred in a room adjacent to the colony room, and the animals were acclimated to the test room overnight prior to testing.

Open Field Task.

Animals were tested in the open field to measure overall locomotor activity and basal anxiety state. In the open field test each rat is placed in a novel environment consisting of an arena measuring 100×100×40 cm. Rats were placed in the middle of the chamber and behavior in the open field was recorded for thirty minutes with a digital camera and measured by software (AnyMaze, Stoelting Co., Inc.). The following parameters were analyzed: a) total distance traveled; b) time in center; c) center entries; and d) number of fecal boli excreted.

Elevated Plus Maze Task.

Animals were tested on the elevated plus maze to measure anxiety-related behavior. The plus-maze is elevated ˜85 cm above the floor and consisted of two open and two closed arms of the same size (50×10 cm). The closed arms were surrounded by walls 40 cm high, and the arms were constructed of black acrylic slabs that radiate from a central platform (10×10 cm) to form a plus sign (Lafayette Instruments, Lafayette, Ind.). Each rat was placed in the central platform facing one of the open arms, and its behavior was recorded during a 5-min testing period with video capture software (AnyMaze). The amount of time spent on open and closed arms and the number of entries into open and closed arms were assessed with video analysis (AnyMaze). In addition, the animals were scored live by the experimenter for time spent rearing, grooming, and number of stretch-attend postures and head dips over the edge of the open arm.

Swim Test.

Each rat was examined in the swim test for depressive-like behaviors. The test was performed over two consecutive days since it is a measure of an adaptive response to a perceived inescapable situation. The first test establishes to the animal that it is an inescapable situation, and the second test measures the animal's degree of passive coping derived from an unwillingness to maintain effort in an inescapable situation. Typical antidepressants limit the amount of passive coping (immobility) and promote the amount of active coping (swimming/climbing). On day one (0800 h to 1300 h), animals were acclimated to the test by placing them in a plexiglass cylindrical container (45 cm×20 cm) filled with 30 cm of fresh water (25° C.) for fifteen minutes, after which they were toweled dried and returned to their home cage. On day two (24 hrs later) the test was performed for a total swim time of five minutes, after which the rats were toweled dried and returned to their home cage. Both trials were recorded by a digital video camera was secured to the ceiling above the cylinders and connected to a laptop. Total time spent swimming and immobile was measured real-time by behavioral software (Anymaze, Stoelting Co.), and scored post hoc by a blinded experimenter from captured video. Swimming was defined as movement of the forelimbs and hind limbs that does not break the surface of the water. Immobility was defined as absence of any movement except for slight movements necessary for the animal to keep its head above water. Climbing was defined as rapid movement of the forelimbs that broke the surface of the water.

Hot Flash Studies.

Animals were anesthetized with intraperitoneal co-injection of urethane (750 mg/kg) and α-chloralose (60 mg/kg). This anesthetic was preferred since it does not typically result in the same degree of hypothermia as is observed with isoflurane, pentobarbital, or ketamine/xylazine. Furthermore, isoflurane has been shown to inhibit CGRP-induced hot flashes. Thermistor probes (ADI Instruments, Colorado Springs, Colo.) were taped to the plantar surfaces of one hind foot. Forty minutes later, after anesthesia-induced hypothermia had stabilized, the basal skin temperature was recorded. Temperature monitoring then occurred at 5-second intervals throughout the remainder of the experiment. Calcitonin gene related peptide (CGRP; 10 μg/kg, i.v.) dissolved in saline was then injected via the tail vein. A maximum of eight animals were tested at the same time due to limitations on instrumental throughput. Cage mates were always tested together in order to avoid the stresses of single-housing and the potential stress hormone effects on body temperature. Therefore only four groups could be tested within any one given day. This resulted in 10 cohorts of 8 animals [one set of eight per day for two weeks (ten weekdays)] as well as a lack of within-day controls for some of the treatment groups. This lack of within-day controls for some groups subsequently caused interference in the final analyses of the hot flash response, so these groups were removed from the hot flash analyses.

### Results

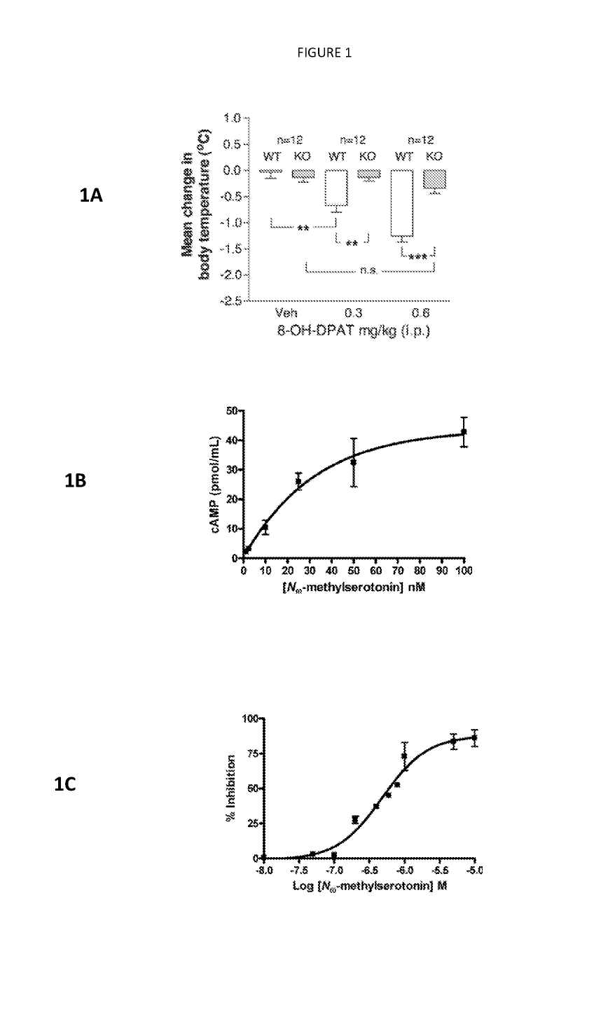

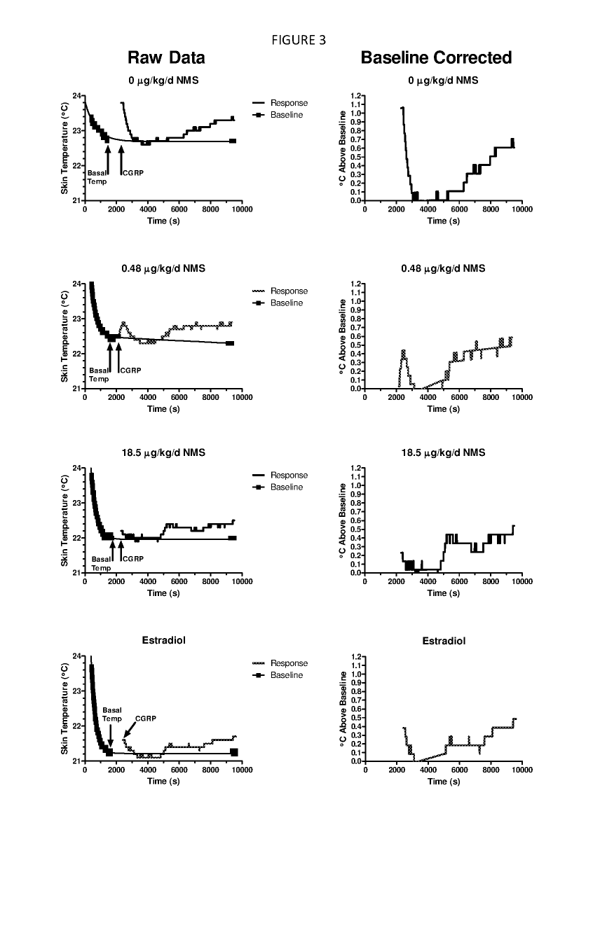

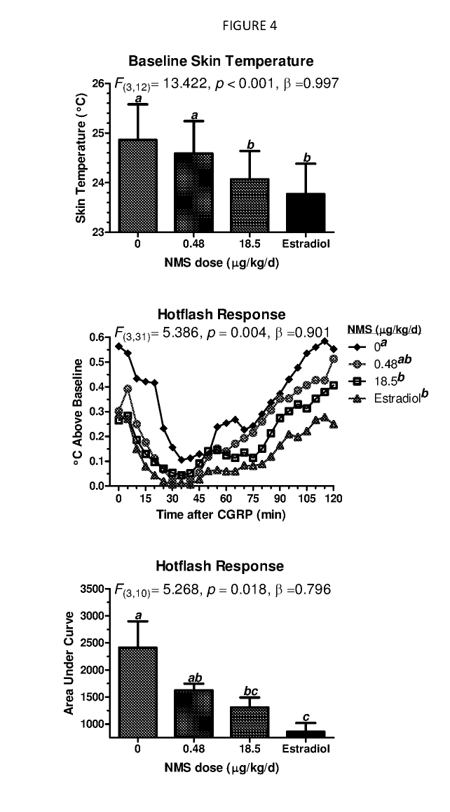

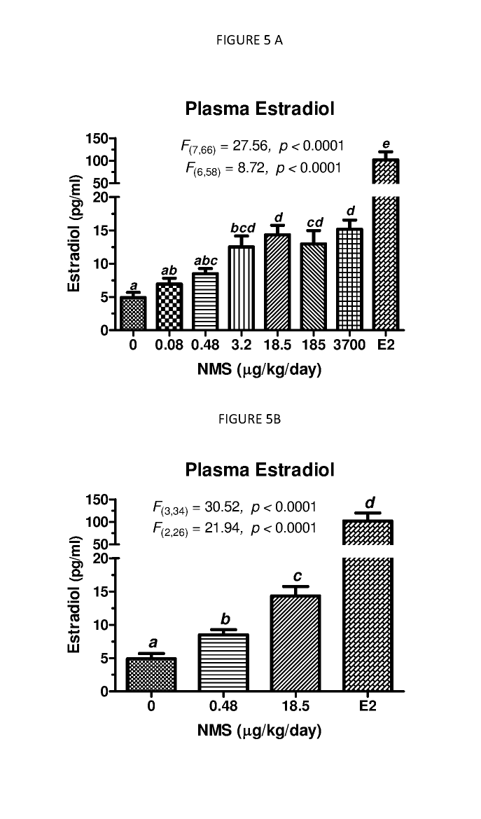

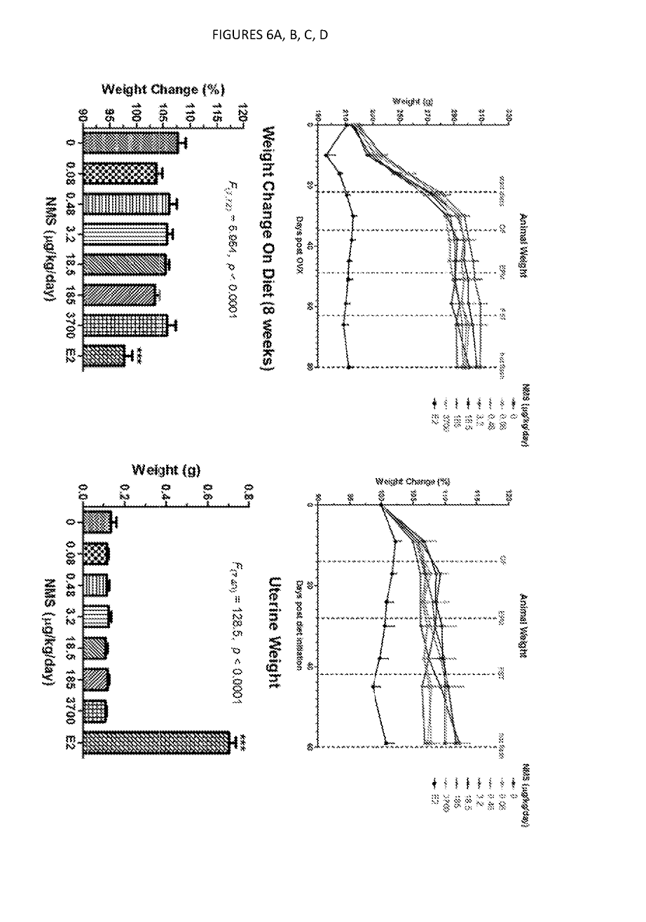

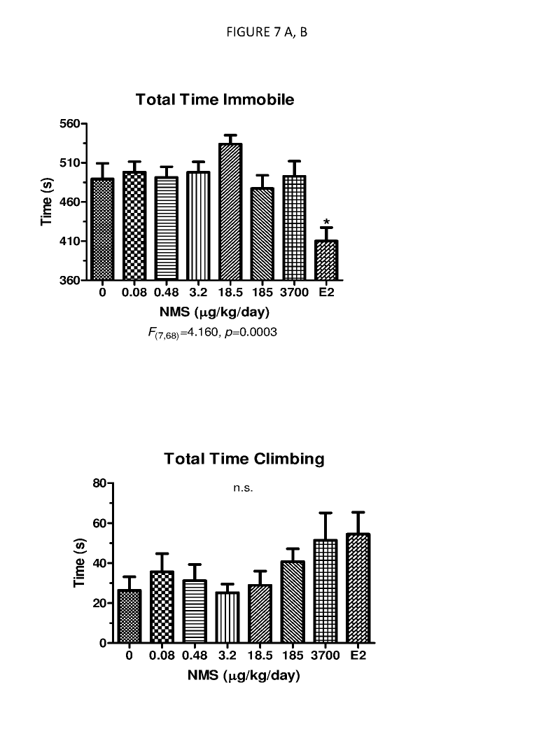

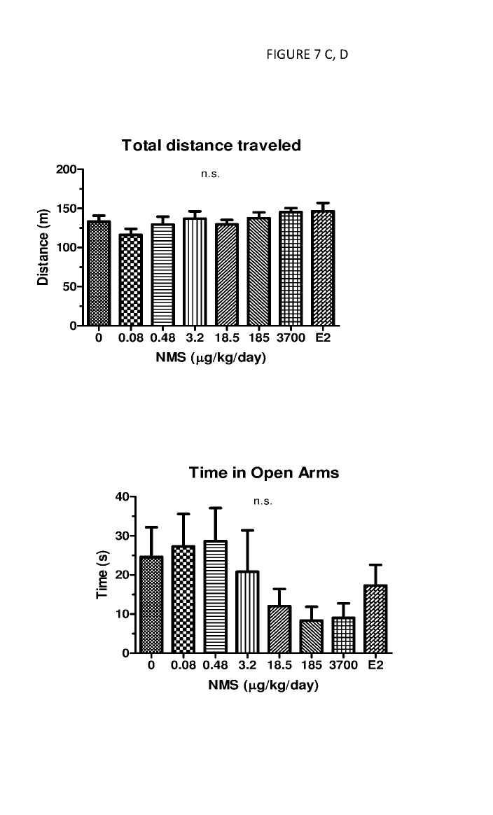

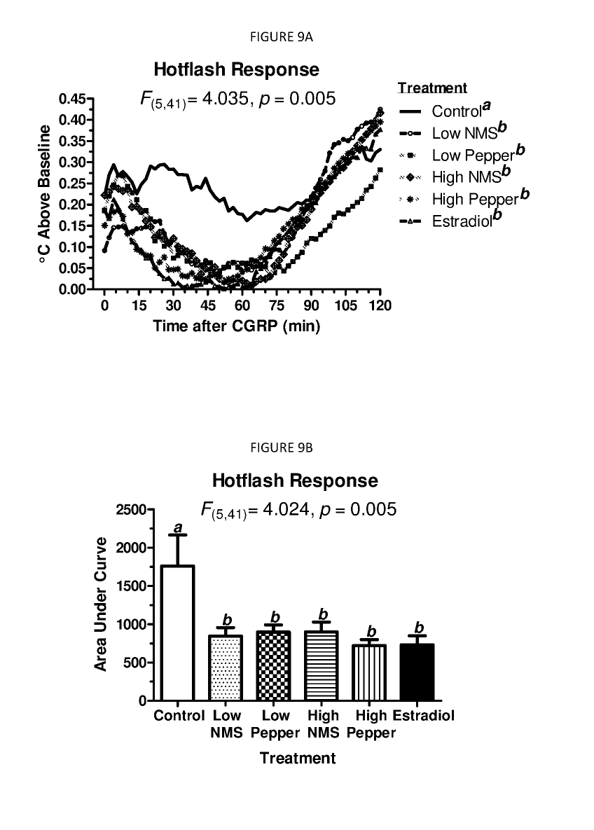

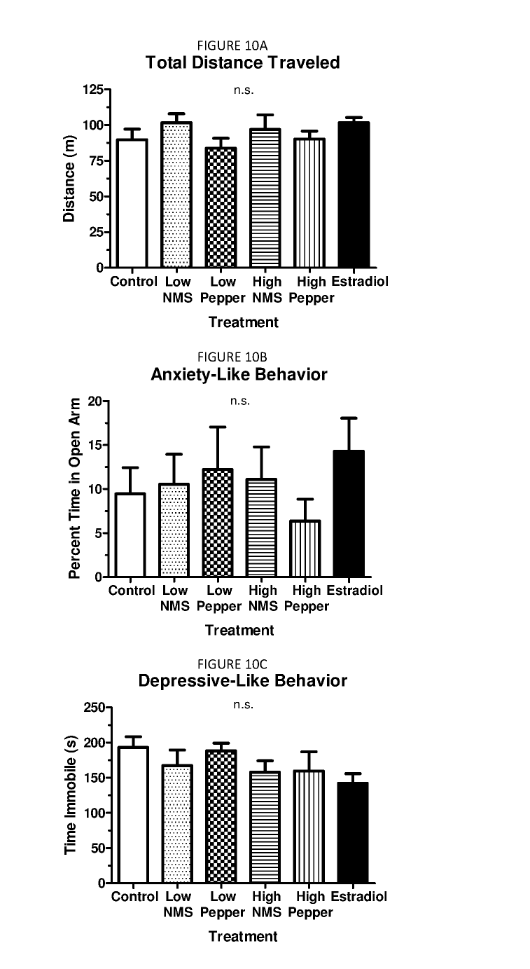

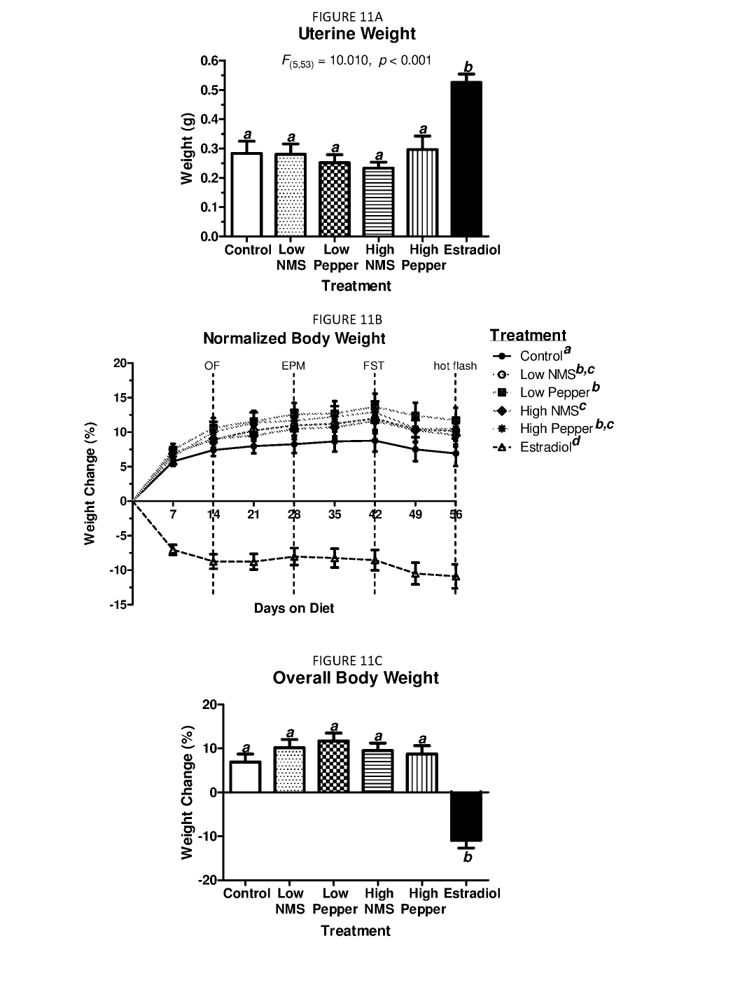

As detailed in FIGS. 1-11;
A) NMS-glucoside from milled Zanthoxylum seed blunted the hot-flash response in a manner similar to estrogen (i.e. estradiol) and to chemically synthesized NMS.
B) Zanthoxylum seed did not affect overall weight gain, uterine growth or mood-related behaviors. These findings were similar to those with chemically synthesized NMS.
C) The human equivalent dose (HED) of NMS for thermoregulatory effects is approximately 150-840 μg/d when the body surface area (BSA) method of calculating HED is used. The data argue that optimization of NMS content in a given product could provide relief from menopausal hot flashes without the risks associated with uterine growth.
D) In a follow-up study, the NMS-glucoside in milled Japanese pepper seed was equivalent to chemically synthesized NMS with an HED=150-540 μg/d (0.7-3.2 g seed/d) when the BSA calculation was used.
E) NMS tripled non-uterine estradiol secretion, which may explain some of the effects of NMS-glucoside from Zanthoxylum seed.

1. The use of n-methylserotonin glucoside (“NMS-glucoside”) or an NMS-precursor in an effective amount to prevent or ameliorate hot flushes associated with menopause.

2. The use according to claim 1, wherein the NMS-glucoside or NMS-precursor is present in a plant source.

3. The use according to claim 1 wherein the plant source is Zanthoxylum piperitum seeds or a Zanthoxylum piperitum seed extract.

4. The use according to claim 1 wherein the Zanthoxylum piperitum seed is a mature seed.

5. The use according to claim 1 wherein the NMS-glucoside is present in a food supplement or nutraceutical.

6. The use according to claim 1 wherein n-methylserotonin (“NMS”) is also used.

7. A method of lessening the frequency of menopausal hot flashes or lessening the severity of menopausal hot flashes comprising administering to a woman in need thereof an effective amount of NMS-glucoside or NMS precursor or mixtures thereof.

8. A method according to claim 7 wherein the NMS-glucoside is from a plant source.

9. A method according to claim 8 wherein the NMS-glucoside is present in Zanthoxylum piperitum seeds or a Zanthoxylum piperitum seed extract.

10. The method according to claim 9 wherein the Zanthoxylum piperitum seed is a mature seed.

11. The method according to claim 7 wherein NMS-glucoside is present in a food supplement or nutraceutical.

12. The method according to claim 7 where NMS is also administered.

13. An extract of a plant which naturally contains NMS-glucoside or NMS which has been standardized to contain an effective amount of NMS-glucoside or NMS such that it lessens the occurrence of hot flashed related to menopause.

14. A food supplement or nutraceutical comprising NMS-glucoside.

15. A food supplement according to claim 14 further comprising NMS.
